(19) United States
(12) Patent Application Publication
(10) Pub. No.: US 2008/0004704 A1
(43) Pub. Date: Jan. 3, 2008

(54) Inter-Vertebral Disc Prosthesis

(76) Inventor: Akiva Raphael Katz, Cape Province (ZA)

Correspondence Address: BIRCH STEWART KOLASCH & BIRCH, PO BOX 747, FALLS CHURCH, VA, 22040-0747, US

(21) Appl. No.: 11/663,651

(22) PCT Filed: Sep. 23, 2005
(86) PCT No.: PCT/IB05/02817

§ 371 Date: May 16, 2007

(30) Foreign Application Priority Data
Sep. 23, 2004 (ZA) 2004/07683

(51) Int. Cl. A61F 2/44

(52) U.S. Cl. 623/17.16; 623/17.15

(57) ABSTRACT

An inter-vertebral disc prosthesis includes a stacked array of spaced elements, adjacent elements being attached to and separated from one another by at least one spacer. The invention extends to an inter-vertebral disc prosthesis characterized in that the prosthesis generates a reactive force generally opposed to and bearing a predetermined relationship with an axial compressive force applied to the prosthesis.

### FIELD OF THE INVENTION

This invention relates to a prosthetic device for the replacement of an inter-vertebral disc.

### BACKGROUND TO THE INVENTION

A number of modalities for treatment of compromised inter-vertebral discs are available, including spinal fusion and the use of inter-vertebral prostheses for the replacement of compromised inter-vertebral discs. Where a prosthesis is used, R should ideally be configured to Incorporate the following features: it should be easily positioned; maintain correct inter-vertebral spacing while allowing for motion, stability, and a range of motion in all planes; demonstrate resistance during flexion and extension that is equivalent to that normally occurring in the natural disc (not to cause undue strain in adjacent levels); be shock absorbent; durable; made from a bio-compatible material; generate minimum wear debris; and be available in a variety of sizes (to accommodate variations in patient height and size), ie it should enable close to normal functioning of the spinal column. Available prostheses are of relatively complex construction and consequently expensive. Many of the available prostheses are unable to meet all of the above criteria.

### OBJECT OF THE INVENTION

It is an object of this invention to provide an inter-vertebral disc prosthesis, which will alleviate, at least partially, the abovementioned problems and achieve, at least in part, the goals set out above.

### SUMMARY OF THE INVENTION

According to a first aspect of the invention there is provided an inter-vertebral disc prosthesis including a stacked array of spaced elements, adjacent elements being attached to and separated from one another by at least one spacer.

The spaced elements may be generally planar discs and may be co-parallel. Instead, the spaced elements may be parallel-planar and kidney-shaped.

The spacers may comprise small discs, a single spacer being located between adjacent spaced elements. Instead, the spacers may comprise sectors of an annulus having the same radius of curvature as a peripheral region of the spaced elements, which they separate. Further, the spacers may be angularly spaced about an axis transverse to the planar spaced elements. Preferably, the spacers are spaced so that when the prosthesis is viewed in plan the spacers are not in register with one another. The spacers may be evenly angularly spaced about the transverse axis. Instead, the spacers may be located in any suitable position with respect to the planar elements and may be in register with one another. The location of the spacers may depend on the anticipated positioning of the centre of load in the particular application for which the prosthesis is intended, in use.

Further, the disc prosthesis may include a pair of end plates. The end plates may be provided by outer elements of the stacked array of spaced elements. Preferably, at least one of the end plates has a convex outer surface. Further preferably, the outer surface of the at least one end plate is surface-textured. It will be appreciated that the end plates as well as the spaced elements of the array may be of varying thicknesses and the number of spaced elements in the array may vary.

The spaced elements may be resiliently flexible to enable the prosthesis to be compressively and extensively deformed in response to forces applied to the outer surfaces of the end plates and to relax to their normal position. In a preferred embodiment of the invention, the prosthesis is adapted to generate a reactive force generally opposed to an axial compressive force applied to the prosthesis, the reactive force being a function of the axial compression of the prosthesis under loading. Further preferably, the reactive force increases as the axial compression of the prosthesis increases. The reactive force may increase smoothly as a function of axial compression of the prosthesis.

The prosthesis may have attachment means for attaching the end plates to neighbouring vertebrae between which the prosthesis is inserted. Then, the attachment means may comprise centrally located protruding formations, which may be saw-toothed or profiled, extending laterally from each end plate. Instead, the protruding formations may be located other than centrally. In one embodiment of the invention the attachment means comprise a plurality of circumferentially spaced pointed formations extending laterally from each end plate. It will be appreciated that any suitable attachment means providing for simple and effective location and attachment to neighbouring vertebra may be provided.

According to a second embodiment of the invention there is provided an inter-vertebral disc prosthesis characterized in that the prosthesis generates a reactive force generally opposed to and bearing a predetermined relationship with an axial compressive force applied to the prosthesis. Preferably, the displacement of the prosthesis under loading bears a predicable and pre-determined relationship to the load applied at a point on an end plate. The reactive force may be a function of the axial compression of the prosthesis under loading. In one embodiment of the invention, the reactive force increases as the axial compression of the prosthesis increases. The reactive force may increase smoothly as a function of axial compression of the prosthesis.

Further, the prosthesis may be of a non-corrosive metal, such as titanium or other bio-compatible material.

### BRIEF DESCRIPTION OF THE DRAWINGS

The invention is now described, by way of example, with reference to the accompanying diagrammatic drawings. In the drawings:

FIG. 1 shows a three-dimensional view of a first embodiment of an inter-vertebral disc prosthesis in accordance with the invention;

FIG. 2 shows a plan view of the prosthesis of FIG. 1;

FIG. 3 shows a side view of the prosthesis of FIG. 1 in a normal unloaded condition;

FIG. 4 shows a side view of the prosthesis of FIG. 1 under a compressive load applied in a first position;

FIG. 5 shows a side view of the prosthesis of FIG. 1 under a compressive load applied in a second position;

FIG. 6 shows a side view of the prosthesis of FIG. 1 under a compressive load applied in a third position;

FIG. 7 shows the prosthesis of FIG. 1 in use between neighbouring vertebrae;

FIG. 8 shows a theoretical plot of the performance of the prosthesis of FIG. 1 under load;

FIG. 9 shows a three dimensional view of a second embodiment of inter-vertebral disc prosthesis in accordance with the invention;

FIG. 10 shows a plan view of the prosthesis of FIG. 9;

FIG. 11 shows a side view of the prosthesis of FIG. 9; and

FIG. 12 shows an end view of the prosthesis of FIG. 9.

### DETAILED DESCRIPTION OF THE DRAWINGS

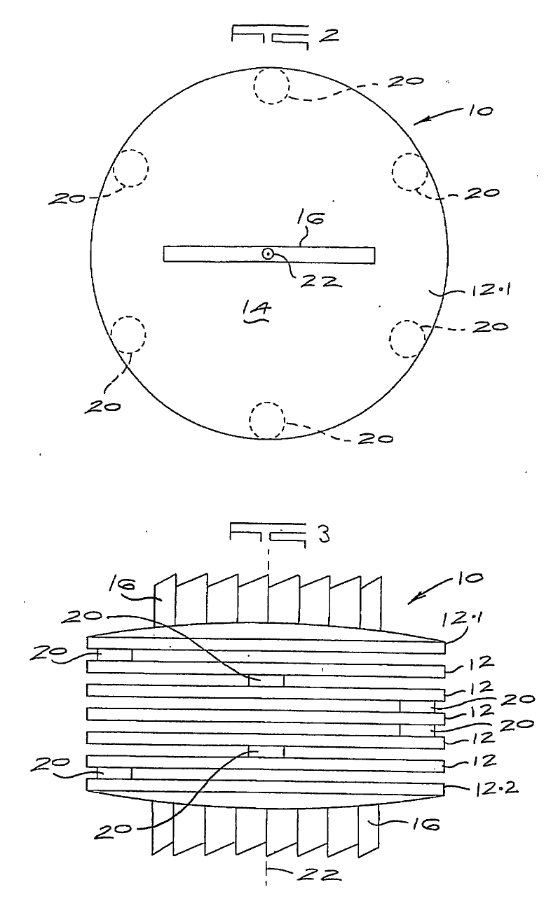

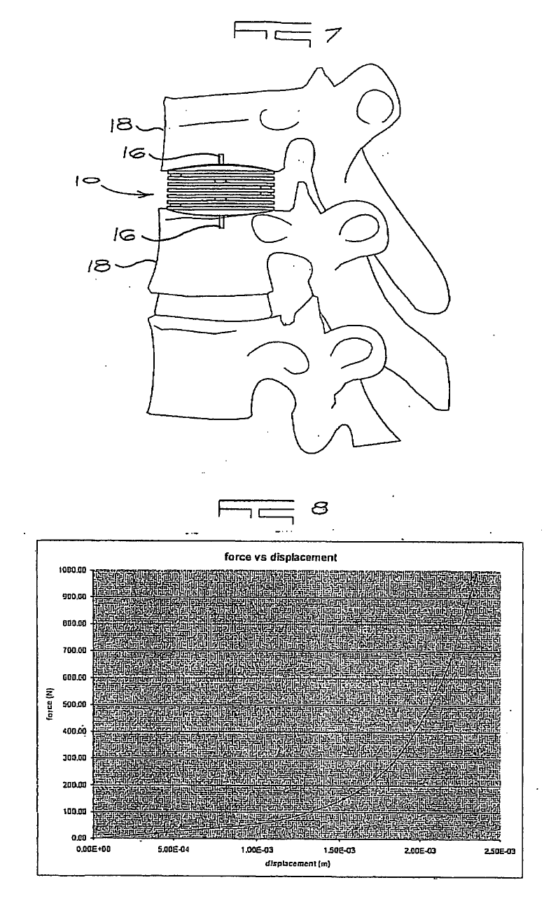

In the drawings, reference numeral 10 generally indicates an inter-vertebral prosthesis in accordance with the invention. The prosthesis comprises an array of seven generally parallel planar titanium circular discs 12. It will be appreciated that the array may comprise any number of discs 12. Outer discs 12.1,12.2 of the array of discs provide end plates. The end plates 12.1,12.2 have a convex outer surface 14, as shown in FIG. 3. It will again be appreciated that only one or none of the end plates 12.1,12.2 may be convex. Attachment formations 16, comprising centrally located profiled members are attached to the end plates 12.1,12.2 and project laterally therefrom. These attachments formations 16 are surgically locatable within adjacent vertebrae 18 when the prosthesis 10 is inserted inter-vertebrally, as shown in FIG. 7.

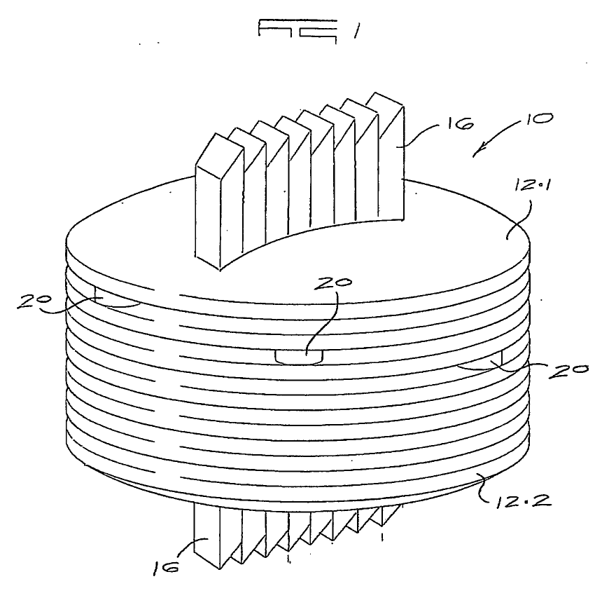

The prosthesis 10 has a series of circular disc-like spacers 20, each spacer 20 being positioned peripherally between adjacent discs 12 of a pair, to both separate and connect the discs 12 to one another. It will be appreciated the spacers 20 may be of any suitable shape. When seen in plan view as in FIG. 2, the spacers 20 are evenly angularly positioned about a notional central lateral axis 22 of the prosthesis 10. It will be appreciated that the spacers 20 may be of any convenient shape and those shown in FIG. 1 are shaped as truncated sectors of a circle having the diameter of the discs 12.

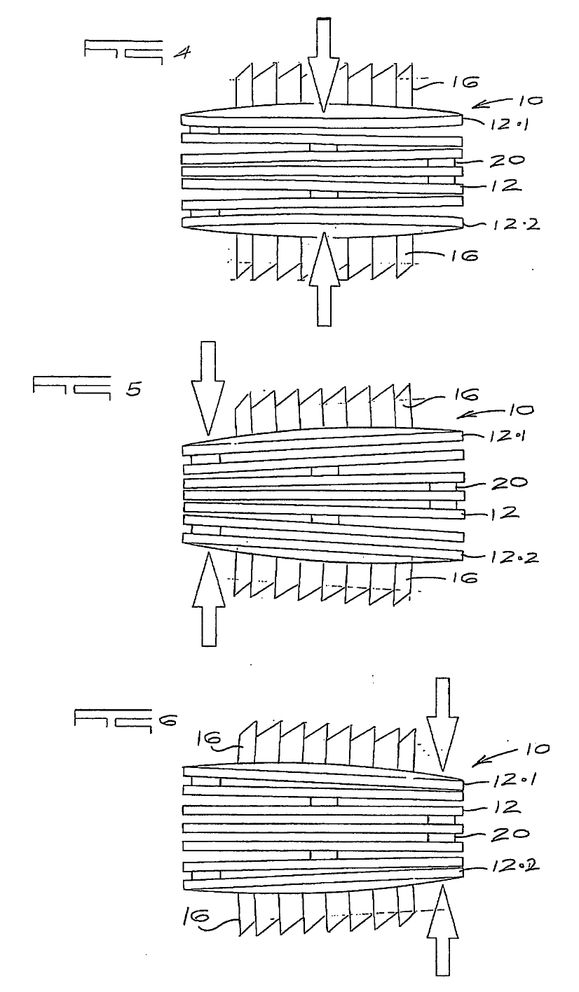

The titanium discs 12 are resiliently flexible to permit compression of the prosthesis 10 in response to axial forces. It will be appreciated that the spacers 20 not being in register, the prosthesis 10 will respond to compressive forces applied anywhere on the end plates 12.1,12.2. FIGS. 4 to 6 show the response of the prosthesis 10 to compressive loading applied respectively at positions spaced 90° apart about the axis 22. It will be appreciated that the response of the prosthesis 10 under loading may be controlled by varying the size, location and number of the spacers 20. Thus, for example, the prosthesis may be made to compress differentially to lateral as opposed to anterior or posterior loading. FIG. 8 shows a plot of force against displacement under loading of the prosthesis 10 for a compressive load applied axially and peripherally to the end plates 12.1, 12.2 of the prosthesis of the drawings. It will be noted that the force and displacement bear a smooth positive, but generally inverse, relationship to one another, as is the case with a natural inter-vertebral disc.

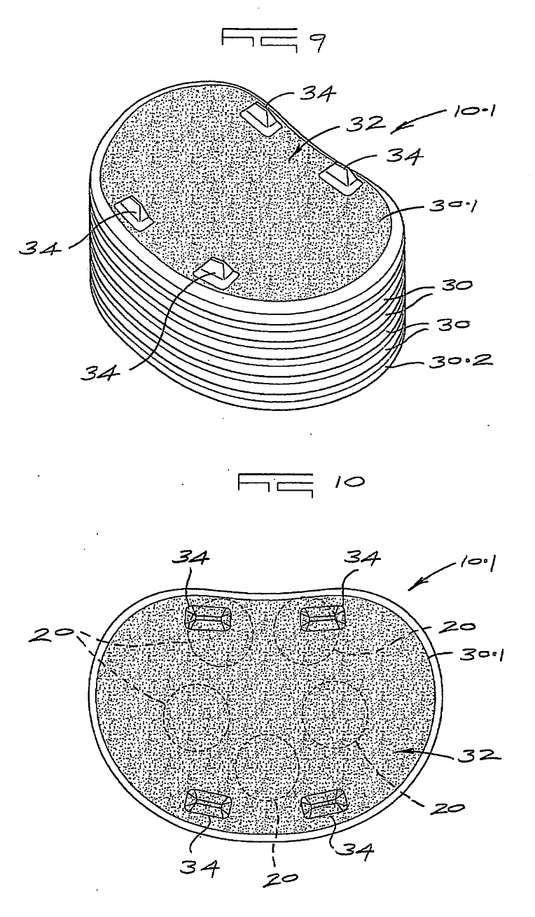

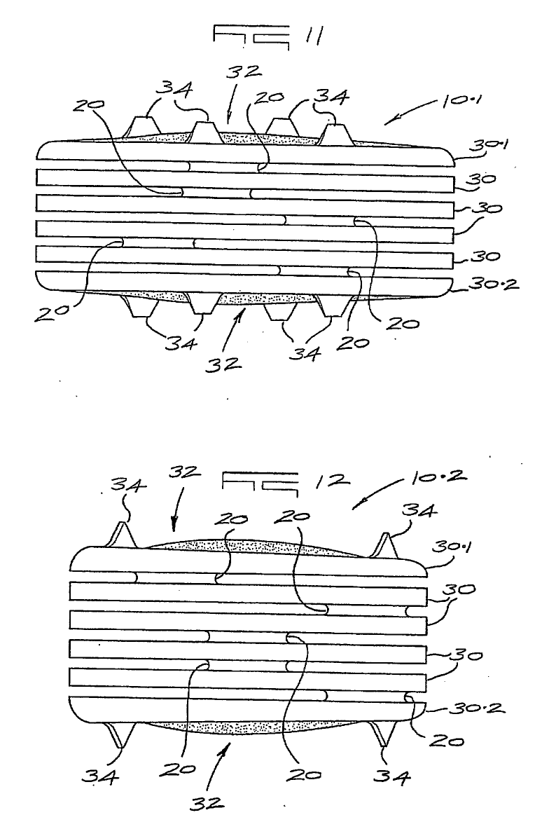

FIGS. 9 to 12 illustrate a second embodiment 10.1 of an inter-vertebral disc prosthesis in accordance with the invention and, with reference to FIGS. 1 to 8, like reference numerals indicate like components, unless otherwise specified.

The second embodiment 10.1 of the prosthesis is similar to the first embodiment 10 and functions in a similar manner. The prosthesis 10.1 comprises a stacked array of six generally planar elements 30 which, as shown in FIG. 10, are generally kidney shaped in plan view. The two outer elements 30.1,30.2 of the stacked array provide end plates for the prosthesis 10.1 and their outer surfaces 32 are convex in form. The convex surfaces 32 are roughly textured to facilitate adhesion with spinal vertebrae between which the prosthesis 10.1 is intended to be located. Further, attachment formations 34, comprising four projecting teeth are located on the outer surfaces 32 of each of the end plates 30.1,30.2. These formations 34 enable the prosthesis 10.1 to engage with adjacent vertebrae facilitating location and attachment thereto. The spacers 20 of the second embodiment 10.1 of the prosthesis comprise small circular discs which are positioned between each adjacent pair of disc elements 30. As may be seen from FIG. 10, in which the disc spacers 20 are indicated in dotted lines, the spacers 20 are angularly spaced about a notional axis which is transverse to the planes of the spaced elements 30. The entire prosthesis 10.1 is of titanium and each of the spaced elements 30 is resiliently flexible and behaves similarly to the elements 12 of the first embodiment 10 of the prosthesis, in that the prosthesis tends, having been deformed by compressive forces applied to the end plates 30.1,30.2 of the prosthesis, to return to its normal state, as illustrated in the drawings. The selection of the thickness of the individual spaced elements 30, the size and spacing of the spacers 20, and the material of the spaced elements 30 enables the resistance to compression under loading and the magnitude of the reactive force urging the prosthesis 10.1 to its normal position and the speed of return to that position to be finely controlled.

By means of the invention, there is provided a relatively simple, effective and hard wearing prosthesis for use in the replacement of compromised vertebral discs. It has been found that the prosthesis responds in a well controlled and predictable manner to applied forces, mimicking the behaviour of natural discs. However, the response of the device to compressive loading may be selectively varied by variations in the positioning of the spacer elements between the spaced elements and the number and size of the spacers used as well as the thickness of the spacers and the spaced elements. Thus, it is possible to configure the prosthesis to deflect less to forces applied at certain positions, as compared with forces applied at other positions on the end plates of the prosthesis.

1-22. (canceled)

23. An inter-vertebral disc prosthesis including a stacked array of spaced generally planar elements, comprising a pair of end plates and at least one intermediate planar element intermediate the end plates, adjacent planar elements being fixedly attached to and separated from one another by at least one spacer, the planar elements being resiliently flexible to enable the device to be compressively and extensively deformed in response to forces applied on outer surfaces of the end plates and to relax to their normal position.

24. An inter-vertebral disc prosthesis as claimed in claim 23, in which the generally planar elements comprise parallel-planar discs.

25. An inter-vertebral disc prosthesis as claimed in claim 23, in which the generally planar elements are parallel-planar and kidney-shaped.

26. An inter-vertebral disc prosthesis as claimed in claim 23, in which the spacers comprise small discs, a single spacer being located between adjacent discs.

27. An inter-vertebral disc prosthesis as claimed in claim 23, in which the spacers comprise sectors of an annulus having the same radius of curvature as a peripheral region of the discs, which they separate.

28. An inter-vertebral disc prosthesis as claimed in claim 23, in which the spacers are angularly spaced about an axis transverse to the planar elements.

29. An inter-vertebral disc prosthesis as claimed in claim 28, in which the spacers are spaced so that when the prosthesis is viewed in plan the spacers are not in register with one another.

30. An inter-vertebral disc prosthesis as claimed in claim 24, in which the end plates are provided by outer disc elements of the stacked array of disc elements.

31. An inter-vertebral disc prosthesis as claimed in claim 30, in which at least one of the end plates has a convex outer surface.

32. An inter-vertebral disc prosthesis as claimed in claim 31, in which the outer surface of the at least one end plate is surface-textured.

33. An inter-vertebral disc prosthesis as claimed in claim 23, in which the prosthesis generates a reactive force generally opposed to an axial compressive force applied to the prosthesis, the reactive force being a function of the axial compression of the prosthesis under loading.

34. An inter-vertebral disc prosthesis as claimed in claim 33, in which the reactive force increases as the axial compression of the prosthesis increases.

35. An inter-vertebral disc prosthesis as claimed in claim 34, in which the reactive force increases smoothly as a function of axial compression of the prosthesis.

36. An inter-vertebral disc prosthesis as claimed in claim 23, which has attachment means for attaching the end plates to neighbouring vertebrae between which the prosthesis is inserted.

37. An inter-vertebral disc prosthesis as claimed in claim 36, in which the attachment means comprise centrally located protruding formations extending laterally from each end plate.

38. An inter-vertebral disc prosthesis as claimed in claim 36, in which the attachment means comprise a plurality of circumferentially spaced pointed formations extending laterally from each end plate.

39. An inter-vertebral disc prosthesis characterized in that the prosthesis generates a reactive force generally opposed to and bearing a predetermined relationship with an axial compressive force applied to the prosthesis.

40. An inter-vertebral disc prosthesis as claimed in claim 39, in which the reactive force is a function of the axial compression of the prosthesis under loading.

41. An inter-vertebral disc prosthesis as claimed in claim 40, in which the reactive force increases as the axial compression of the prosthesis increases.

42. An inter-vertebral disc prosthesis as claimed in claim 41, in which the reactive force increases smoothly as a function of axial compression of the prosthesis.
